(19) United States
(12) Patent Application Publication
(10) Pub. No.: US 2006/0035954 A1
(43) Pub. Date: Feb. 16, 2006

(54) Ammonolysis process for the preparation of intermediates for DPP IV inhibitors

(76) Inventors: Padam N. Sharma, Manlius, NY (US); Gabriel M. Galvin, Maple Valley, WA (US); Susan D. Boettger, Fayetteville, NY (US); Saibaba Racha, Smithtown, NY (US); Jingyang Zhu, Monmouth Junction, NJ (US); Jack Melton, Fayetteville, NY (US); Boguslaw M. Mudryk, East Windsor, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/199,539

(22) Filed: Aug. 8, 2005

Related U.S. Application Data
(60) Provisional application No. 60/600,510, filed on Aug. 11, 2004.

(51) Int. Cl. A61K 31/403; C07D 209/52

(52) U.S. Cl. 514/412; 548/537; 548/452

(57) ABSTRACT

A process is provided for preparing the intermediate A in accordance with the following reaction sequence 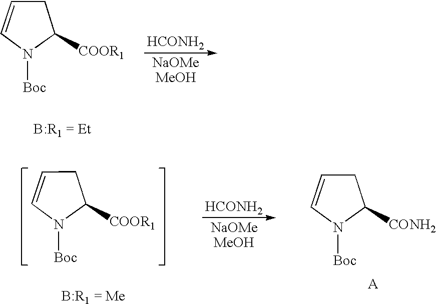 The intermediate A is used in preparing DPP IV inhibitors which are useful in treating diabetes.

This application claims a benefit of priority from U.S. Provisional Application No. 60/600,510, filed Aug. 11, 2004, the entire disclosure of which is herein incorporated by reference.

### FIELD OF THE INVENTION

The present invention relates to an ammonolysis process for the preparation of intermediates useful in preparing dipeptidyl peptidase (DPP) IV inhibitors and to a method for preparing DPP IV inhibitors employing such intermediates.

### BACKGROUND OF THE INVENTION

U.S. Provisional Application No. 60/431,814 filed Dec. 9, 2002 and its corresponding non-provisional application Ser. No. 10/716,012 filed Nov. 18, 2003 disclose a method for preparing the intermediate (5S)-5-aminocarbonyl-4,5-dihydro-1H-pyrrole-1-carboxylic acid, 1-(1,1-dimethylethyl) ester (Formula A) 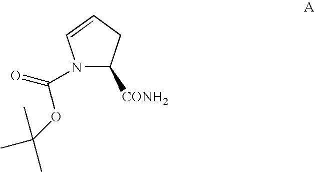
from 4,5-dihydro-1H-pyrrole-1,5-dicarboxylic acid, 1-(1,1-dimethylethyl)-5-ethyl or 5-methyl ester (Formula B) 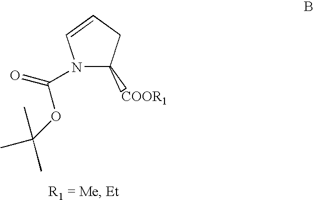
in a three-step procedure by hydrolyzing B with an alkali metal hydroxide such as sodium hydroxide or lithium hydroxide, in the presence of dicyclohexylamine (DCHA) to form the DCHA acid salt, treating the salt with mesyl chloride or with 4-(4,6-dimethoxy-1,3,5-triazin-2-yl)-4-methylmorpholinium chloride (DMT-MM), to form the corresponding activated ester (mesyl or DMT ester) and treating the ester with ammonia to form the Formula A intermediate.

The Formula A intermediate is useful in preparing the dipeptidyl peptidase IV inhibitor (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile M 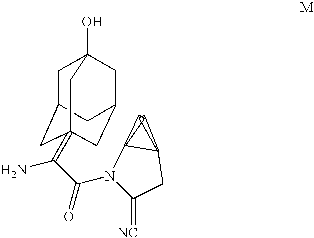
(disclosed in U.S. Pat. No. 6,395,767 which is incorporated herein by reference) as discussed in U.S. Provisional Application No. 60/431,814 and its corresponding non-provisional application Ser. No. 10/716,012 which is useful in treating diabetes.

### BRIEF DESCRIPTION OF THE INVENTION

In accordance with the present invention, a method is provided for the preparation of an intermediate of the structure A 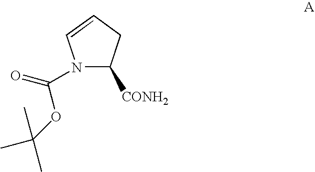
(5S)-5-aminocarbonyl-4,5-dihydro-1H-pyrrole-1-carboxylic acid, 1-(1,1-dimethylethyl) ester, also referred to as (2S)-2-aminocarbonyl-2,3-dihydro-1H-pyrrole-1-carboxylic acid, 1-(1,1-dimethylethyl) ester, which is useful in preparing the dipeptidyl peptidase IV inhibitor (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile disclosed in U.S. Pat. No. 6,395,767.

The method of the invention for making intermediate A includes the step of providing the compound (5S)-4,5-dihydro-1H-pyrrole-1,5-dicarboxylic acid, 1-(1,1-dimethylethyl)-5-ethyl or 5-methyl ester having the structure B 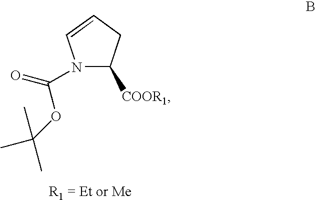
(also referred to as (2S)-1H-pyrrole-1,2-dicarboxylic acid, 2,3-dihydro-, 1-(1,1-dimethylethyl)-2-ethyl or 2-methyl ester, respectively), and subjecting compound B to ammonolysis in the presence of an ammonia source capable of converting an ester to an amide, such as formamide, ammonia gas, ammonium carbamate, ammonium formate, ammonium phosphate, and ammonium acetate, as well as ammonia sources as set out hereinafter, preferably formamide, and base such as sodium methoxide, potassium methoxide, lithium methoxide, magnesium methoxide, magnesium ethoxide, sodium ethoxide, potassium ethoxide, lithium ethoxide, sodium propoxide, potassium propoxide, lithium propoxide, sodium t-butoxide, potassium t-butoxide, lithium t-butoxide, as well as bases as set out hereinafter, preferably sodium methoxide, in the presence of solvent such as formamide, dichloromethane, methanol, ethanol, isopropanol, n-propanol, n-butanol, t-butanol or ethylene glycol, as well as solvents set out hereinafter, preferably methanol, to form the intermediate A.

In carrying out the ammonolysis of compound B, the reaction will be conducted under mild conditions such as at a temperature within the range from about −100 to about 200° C., preferably from about 15 to about 25° C.

The ammonia source will be employed in a molar ratio to the base within the range from about 1:1 to about 200:1, preferably from about 1:1 to about 15:1.

In a preferred embodiment of the process of the invention, the starting intermediate B will be dissolved in a solvent or without solvent, preferably methanol. Base, preferably sodium methoxide, in a solvent or without solvent, preferably methanol, is added to the ammonia source, preferably formamide. The resulting solution is added to the solution of intermediate B, and the reaction is allowed to proceed without heating. The reaction is monitored (such as by HPLC) for presence of starting ethyl ester compound B or its corresponding methyl ester B 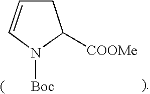
Should either be present, additional ammonia source, preferably formamide, in base, preferably sodium methoxide, is added and the reaction is allowed to proceed until substantially complete.

In a preferred embodiment of the invention, the process of the invention for preparing intermediate A is carried out as a one-pot process where no intermediates, such as methyl ester B (R₁=Me), are isolated.

In another aspect of the present invention, the Formula A amide is an intermediate used in forming the hydrochloride salt or MSA salt of the fragment (1S,3S,5S)-2-azabicyclo[3.1.0]hexane-3-carboxamide (Formula J) 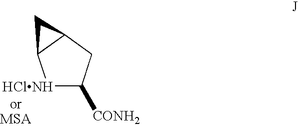
as described in detail in U.S. Provisional Application No. 60/431,814 filed Dec. 9, 2002 and its corresponding non-provisional application Ser. No. 10/716,012 filed Nov. 18, 2003 (Attorney Docket LA0084) and as set out below.

The fragment (1S,3S,5S)-2-azabicyclo[3.1.0]hexane-3-carboxamide (Formula J) is used in the production of the dipeptidyl peptidase IV inhibitor (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile.

These inhibitors are ultimately formed from the coupling of two fragments, the Formula J compound and BOC-protected (αS)-α-amino-3-hydroxytricyclo[3.3.1.1 ^{3,7}]decane-1-acetic acid as depicted in Formula VI, 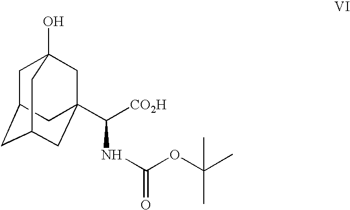
to form free base M, 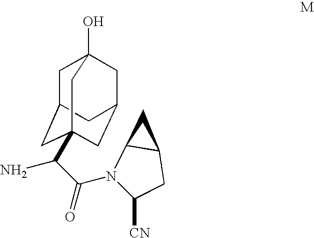
or the monohydrate M′ thereof

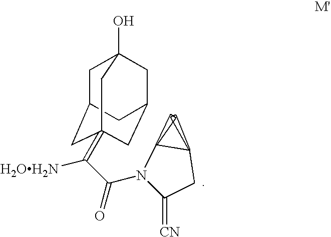

Cyclopropyl-fused pyrrolidine-based compounds such as (1S,3S,55)-2-[(2S)-2-amino-2-(3-hydroxytricyclo[3.3.1.1 ^{3,7}]dec-1-yl)-1-oxoethyl]-2-azabicyclo[3.1.0]hexane-3-carbonitrile, free base (M) and monohydrate thereof (M′) are dipeptidyl peptidase IV inhibitors useful in the treatment of diabetes and complications thereof, and related diseases as disclosed hereinafter.

The ammonolysis process of the invention provides an efficient means for obtaining compounds of Formula A, which are intermediates in the preparation of dipeptidyl peptidase IV inhibitors. Reduction or elimination of undesirable byproducts (which may be obtained employing prior art processes), preservation of enantiopurity (as opposed to chemical racemization) and shortening cycle times in an environmentally friendly manner (waste reduction) may be achieved by employing the ammonolysis method of the present invention. Another benefit derived from the ammonolysis method of the invention is that the method may be conducted employing relatively mild reaction conditions, namely temperatures varying from about 10° C. to about 70° C.

### DETAILED DESCRIPTION OF THE INVENTION

Ammonolysis of 4,5-dihydro-1H-pyrrole-1,5-dicarboxylic acid, 1-(1,1-dimethylethyl)-5-ethyl or 5-methyl ester B (R₁=Me or Et), to (5S)-5-aminocarbonyl-4,5-dihydro-1H-pyrrole-1-carboxylic acid, 1-(1,1-dimethylethyl) ester A in accordance with the present invention is depicted in Scheme I.

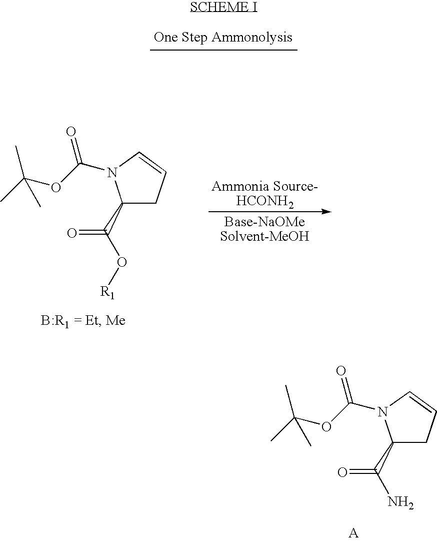

The reaction is carried out at a temperature within the range from about −100 to about 200° C., preferably from about 15 to about 25° C., for a period within the range from about 0.5 to about 72 hours, preferably from about 3 to about 5 hours. Solvents which may be optionally, but preferably, employed herein include but are not limited to formamide, dichloromethane, toluene, chloroform, THF, acetonitrile, methyl acetate, ethyl acetate, isopropyl acetate, propyl acetate, butyl acetate, acetone, methyl isobutyl ketone, methyl ethyl ketone, 1,2-dimethoxyethane, 2-methyltetrahydrofuran, 1,4-dioxane, methyl t-butyl ether (MTBE), chlorobenzene, xylenes, heptane, hexanes, cyclohexane, cyclohexanone, DMF, dimethyl sulfoxide, N-methylpyrrolidinone, MTBE, methanol, ethanol, isopropanol, n-propanol, n-butanol or t-butanol, and ethylene glycol, preferably methanol.

The solvent may be added to compound B and/or ammonia source and/or base prior to or during the reaction. Examples of ammonia sources which may be employed herein include, but are not limited to, formamide, ammonia gas, ammonium carbamate, ammonium formate, ammonium phosphate, ammonium acetate, ammonium fluoride, ammonium bromide, ammonium chloride, ammonium iodide, ammonium iodate, ammonium carbonate, ammonium citrate, ammonium chromate, ammonium dichromate, ammonium hydroxide, ammonium lactate, ammonium molybdate, ammonium nitrate, ammonium oxalate, ammonium sulfate, ammonium sulfide, ammonium tartrate, ammonium triflate, ammonium thiocyanate, ammonium dihydrogen phosphate, urea, methyl carbamate, ethyl carbamate, propyl carbamate or t-butyl carbamate, preferably formamide.

Bases which may be employed herein include, but are not limited to, alkali metal alkoxides such as sodium alkoxide, potassium alkoxide, magnesium alkoxide or lithium alkoxide, alkali metal methoxide, alkali metal ethoxide, alkali metal propoxide or alkali metal butoxide, and include, but are not limited to, sodium methoxide, potassium methoxide, lithium methoxide, magnesium methoxide, magnesium ethoxide, sodium ethoxide, potassium ethoxide, lithium ethoxide, sodium propoxide, potassium propoxide, lithium propoxide, sodium t-butoxide, potassium t-butoxide, lithium t-butoxide, sodium hydride, potassium hydride, pyridine, triethylamine, diethylamine, diisopropylamine, diisopropylethylamine (Hunig's base), 1,8-diazabicyclo[5.4.0]undec-7-ene (DBU), 1,5-diazabicyclo[4.3.0]non-5-ene (DBN), or 1,4-diazabicyclo[2.2.2]octane (DABCO), KHCO₃, NaHCO₃, Na₂CO₃, K₂CO₃, Li₂CO₃, BaCO₃, CaCO₃, Cs₂CO₃, MgCO₃, KOH, NaOH, or LiOH either alone or with catalytic amounts of NiCl₂, CeCl₃, MgBr₂, Sc(III)(OTf)₃, Fe(OAc)₂, Cu(I)SCN, basic alumina, AgOAc, MnCl₂, Cu(OAc)₂, Co(OAc)₂, Zn(OAc)₂, Pd(OAc)₂, FeCl₃, Ti(OPr)₄, tetrabutylammonium chloride, tetrabutylammonium bromide, dodecanethiol or 2-hydroxypyridine. The base is preferably sodium methoxide.

The intermediate A may be separated from the reaction mixture by treating the reaction mixture with ammonium chloride, toluene and water, and solvent may be removed under reduced pressure.

The starting compound B (R₁=Et) is prepared as described in U.S. Provisional Application No. 60/431,814, filed Dec. 9, 2002 and its corresponding non-provisional application Ser. No. 10/716,012 filed Nov. 18, 2003 which is incorporated herein by reference and in Scheme II set out below.

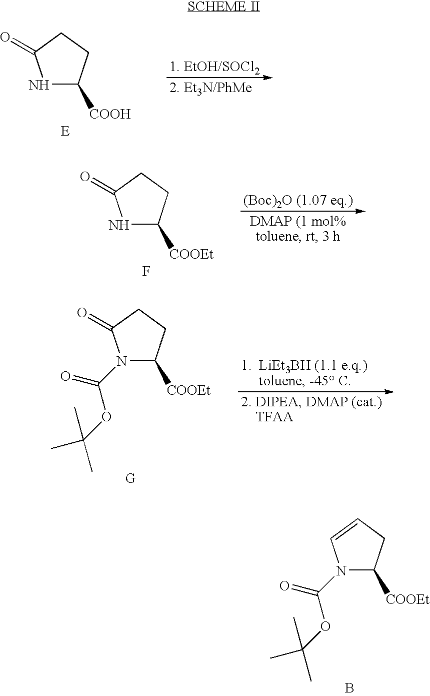

As shown in Scheme II, L-pyroglutamic acid (Formula E) is first esterified to produce the L-pyroglutamic acid ethyl ester (Formula F). This L-pyroglutamic acid ethyl ester is then BOC-protected on the nitrogen to produce (5S)-2-oxopyrrolidine-1,5-dicarboxylic acid, 1-(1,1-dimethylethyl)-5-ethyl ester (Formula G). SuperHydride reduction and elimination is then performed to form 4,5-dihydro-1H-pyrrole-1,5-dicarboxylic acid, 1-(1,1-dimethylethyl)-5-ethyl ester (Formula B) which is then subjected to one step ammonolysis, in accordance with the method of the invention, to form compound A.

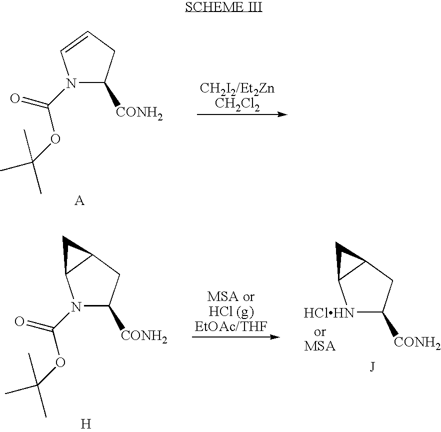

Compound A is employed to form intermediate J as shown in Scheme III above and as described below. As seen in Scheme III, (5S)-5-aminocarbonyl-4,5-dihydro-1H-pyrrole-1-carboxylic acid, 1-(1,1-dimethylethyl) ester (Formula A) is cyclopropanated via the Simmons-Smith reaction to produce (1S,3S,5S)-3-aminocarbonyl-2-azabicyclo[3.1.0]hexane-2-carboxylic acid, 1,1-dimethylethyl ester (Formula H). BOC is then removed resulting in formation of an acid salt such as the hydrochloride salt or the methanesulfonic acid salt of the fragment (1S,3S,5S)-2-azabicyclo[3.1.0]hexane-3-carboxamide (Formula J).

The compound J is used to prepare the dipeptidyl peptidase IV inhibitor formula M compound in accordance with the following reaction Scheme IV which is described in detail in U.S. Provisional Application No. 60/431,814 filed Dec. 9, 2002 and its corresponding non-provisional application Ser. No. 10/716,012 filed Nov. 18, 2003 which is incorporated herein by reference.

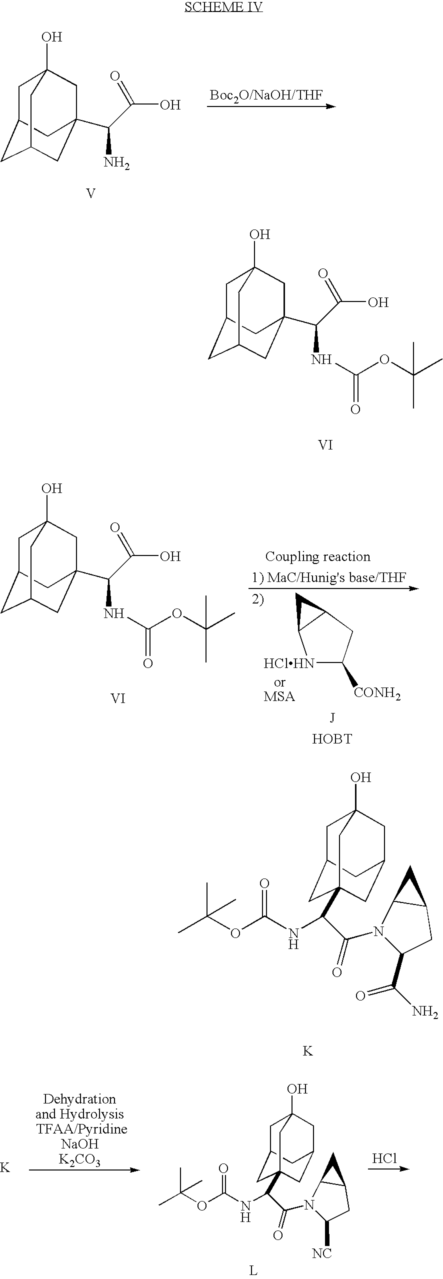

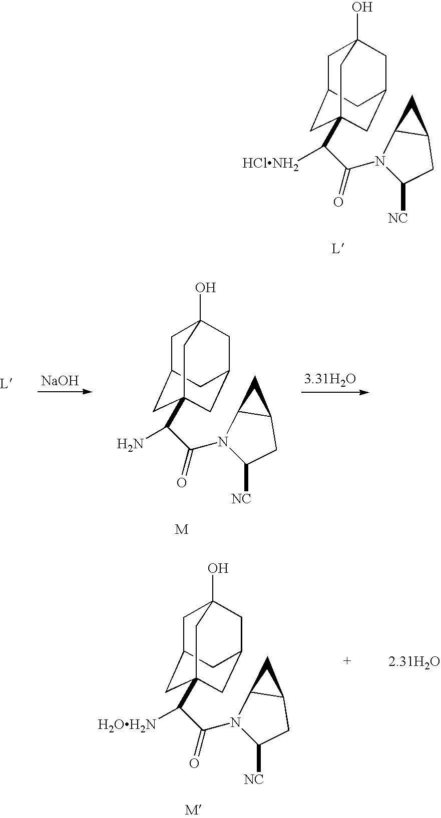

As shown in Scheme IV, the fragment (αS)-(α-amino-3-hydroxytricyclo [3.3.1.1^{3,7}]decane-1-acetic acid (Formula V) is first BOC protected to produce ((αS)-α[[(1,1-dimethylethoxy)carbonyl]amino]-3-hydroxytricyclo [3.3.1.1^{3,7}] decane-1-acetic acid (Formula VI) by treating V with BOC₂O in the presence of base such as sodium hydroxide, acidifying and extracting free acid VI into ethyl acetate (EtOAc). Alternatively, in place of ethyl acetate, isopropyl acetate/heptane (2.25:1) may be employed to crystallize out free acid VI.

The compound V may be prepared as described in U.S. Pat. No. 6,395,767 to Hamann et al. and in U.S. application Ser. No. 10/716,012 filed Nov. 18, 2003 and Provisional Application No. 60/561,986 filed Apr. 14, 2004, all of which are incorporated herein by reference.

A solution of Formula VI compound in an appropriate organic solvent such as tetrahydrofuran (THF) (cooled to a temperature within the range from about 20 to about 30° C.) is treated with methanesulfonyl chloride (MsCl), and Hunig's base (diisopropylethylamine or DIPEA) to form the corresponding methanesulfonic ester of VI.

A coupling reaction is then used to couple the methanesulfonic ester of (αS)-α[[(1,1-dimethylethoxy)carbonyl]amino]-3-hydroxytricyclo[3.3.1.1 ^{3,7}]decane-1-acetic acid (Formula VI), to (1S,3S,5S)-2-azabicyclo[3.1.0]hexane-3-carboxamide (Formula J, methanesulfonic acid salt or HCl salt), in the presence of 1-hydroxybenzotriazole (HOBT) or other known coupling agent to produce 3-(aminocarbonyl)-(αS)-α-(3-hydroxytricyclo[3.3.1.1^{3,7}]dec-1-yl)-β-oxo-(1S,3S,5S)-2-azabicyclo[3.1.0]hexane-2-ethanecarbamic acid, 1,1-dimethylethyl ester (Formula K). Formula K compound is subjected to dehydration by treating compound K with an organic base such as pyridine or triethylamine, and trifluoroacetic anhydride or other dehydrating agent such as phosphorus oxychloride (POCl₃) or cyanuric chloride, and then subjecting the reaction to hydrolysis by heating to from about 25 to about 40° C. and treating with sodium hydroxide or other strong base such as KOH or LiOH to form 3-cyano-(αS)-α-(3-hydroxytricyclo[3.3.1.1^{3,7}]dec-1-yl)-β-oxo-(1S,3S,5S)-2-azabicyclo[3.1.0]hexane-2-ethanecarbamic acid, 1,1-dimethylethyl ester (Formula L).

The free base monohydrate M′ may be formed from the BOC-protected intermediate L as follows.

BOC-protected intermediate L is treated with concentrated hydrochloric acid in the presence of dichloromethane and methanol while maintaining reaction temperature within the range from about 20 and 25° C., to form hydrochloride salt L′. Hydrochloride salt L′ is treated with sodium hydroxide or other strong base to form the free base M. Free base M is then treated with water to form the free base monohydrate M′.

Dipeptidyl peptidase IV inhibition produced by using the compounds and methods of the present invention are useful in the treatment of diabetes and complications thereof, hyperglycemia, Syndrome X, hyperinsulinemia, obesity, and atherosclerosis and related diseases, as well as immunomodulatory diseases and chronic inflammatory bowel disease.

The following Examples represent preferred embodiments of the invention.

### EXAMPLE 1

### (5S)-5-Aminocarbonyl-4,5-dihydro-1H-pyrrole-1-carboxylic acid, 1-(1,1-dimethylethyl) ester (A)

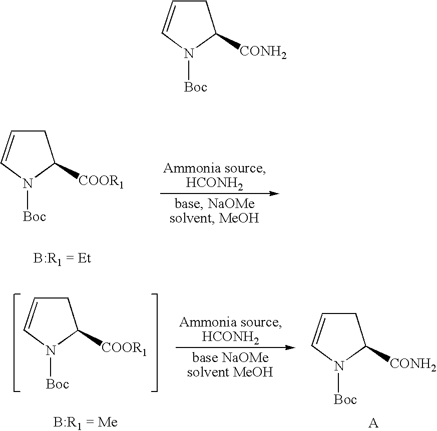

Crude 4,5-dihydro-1H-pyrrole-1,5-dicarboxylic acid, 1-(1,1-dimethylethyl)-5-ethyl ester 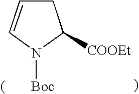
(B, R₁=Et) (5 g) was dissolved in methanol (30 mL). A 25 weight % sodium methoxide solution in methanol (9.0 g, 2 equiv) was added to formamide (13.97 g, 15 equiv) in a separate vessel and the solution was added dropwise to the reaction mixture. The reaction mixture was stirred for 3.5 h at room temperature and monitored by HPLC. HPLC showed the presence of the starting ethyl ester B (R1=Et) or the corresponding methyl ester B (R₁=Me) in the reaction mixture. Additional formamide (0.9 g) in 25 weight % sodium methoxide (4 g) was added to the reaction mixture and stirring was continued for additional 1 h. HPLC showed the absence of the starting ethyl ester B (R₁=Et) or the corresponding methyl ester B (R₁=Me) in the reaction mixture. The reaction mixture was diluted by the addition of saturated aqueous ammonium chloride (10 mL) followed by toluene (100 mL) and water (50 mL). The methanol was removed under reduced pressure. The organic layer was separated and the aqueous layer was re-extracted with toluene (2×50 mL). The combined organic layer was washed with 2:1 brine-water (15 mL), dried over sodium sulfate and filtered. The filtrate was concentrated under reduced pressure to give 2.1 g of the title compound A as an oily residue. It contained 5.9% of the R enantiomer of the title compound by chiral HPLC.

### EXAMPLE 2

### Preparation of the Dipeptidyl Peptidase IV inhibitor (1S,3S,5S)-2-[(2S)-2-amino-2-(3-hydroxytricyclo[3.3.1.1^{3,7}]-dec-1-yl)-1-oxoethyl]-2-azabicyclo-[3.1.0]hexane-3-carbonitrile (Formula M)

### A. Cyclopropanation of (5S)-5-aminocarbonyl-4,5-dihydro-1H-pyrrole-1-carboxylic acid, 1-(1,1-dimethylethyl) ester (Formula A)

Reactor A, was charged with Formula A compound (4 kg) dissolved in dichloromethane (18.0 L) and maintained at 20° C. A second reactor, Reactor B, was charged with dichloromethane (18.00 L) and cooled to −30° C. Reactor B was then charged with dimethoxyethane (DME) (3.36 kg), followed by a 30% solution of diethylzinc (15.36 kg) in toluene, while maintaining the temperature between −30 and −25° C. Reactor B was then charged with diiodomethane (19.99 kg) while maintaining the reaction temperature between −30 and −25° C. After complete addition of the diiodomethane, the mixture was stirred for 45 min at −30 to −25° C. This mixture was then charged to Reactor A via a cooled pipe (−20 to −25° C.). Charging was performed slowly in portions of approximately 5% so that the temperature of the mixture in Reactor A was maintained between 22 and 24° C. until the reaction was completed. Following completion of the reaction, the mixture in Reactor A was cooled to 5 to 10° C. The reaction mixture was then slowly charged with saturated bicarbonate solution (21.6 L) in a manner so that the reaction temperature did not exceed 15° C. Following this addition, the reaction mixture was stirred for at least 1 h while a precipitate formed. The suspension was filtered. The resulting filter cake was transferred back to the vessel, slurried again with dichloromethane (14.4 L) for 30 min, and re-filtered. Following this second filtration, the filter cake was washed with additional dichloromethane (7.2 L). The filtrates were then separated into aqueous and organic phases and the organic phase was washed with half-saturated brine (21.6 L). Solvent was then removed by vacuum at a maximum temperature of 30° C. and exchanged with heptane. A slurry of crude product in heptane was obtained. Final volume of the suspension after solvent exchange was 14.4 L. The crude product was isolated by filtration. The filter cake was washed with heptane (2.9 L) and then dried under vacuum to a constant weight. The crude yield was 2.76 kg (12.2 mol, 72%) of (1S,3S,5S)-3-aminocarbonyl-2-azabicyclo[3.1.0]hexane-2-carboxylic acid, 1,1-dimethylethyl ester (Formula H). To purify, the crude material was slurried in an eight-fold amount of a 1:1 mixture of butyl acetate/heptane at 20 to 22° C. for 4 h. The material was filtered and the filter cake was washed with approximate 1 volume of heptane. The yield was 2.11 kg (9.33 mol, 55%) of (1S,3S,5S)-3-aminocarbonyl-2-azabicyclo[3.1.0]hexane-2-carboxylic acid, 1,1-dimethylethyl ester (Formula H).

### B. Deprotection of (1S,3S,5S)-3-aminocarbonyl-2-azabicyclo[3.1.0]hexane-2-carboxylic acid, 1,1-dimethylethyl ester (Formula H) to form (1S,3S,5S)-2-azabicyclo[3.1.0]hexane-3-carboxamide (Formula J)

A 100-mL, two-necked flask equipped with a mechanical stirrer and a thermocouple was charged with (1S,3S,5S)-3-aminocarbonyl-2-azabicyclo[3.1.0]hexane-2-carboxylic acid, 1,1-dimethylethyl ester (Formula H) (5.0 g, 22.1 mmol) and THF (20 mL). HCl (2.5 M in EtOAc, 25 mL, 62.5 mmol) was then added to the suspension. The resulting solution was stirred at room temperature for 18 h during which time precipitation was observed. Completion of the reaction was monitored by HPLC. Methyl t-butyl ether (MTBE, 30 mL) was added to the suspension and stirring was continued for an additional 30 min. The suspension was then filtered under N₂ protection to produce a white solid that was washed with MTBE (20 mL). The solid was dried in an oven under reduced pressure for 48 h to afford the hydrochloride salt of (1S,3S,5S)-2-azabicyclo[3.1.0]hexane-3-carboxamide (Formula J; 3.6 g, 100%).

### C. BOC Protection of (αS)-α-amino-3-hydroxytricyclo[3.3.1.1^{3,7}] decane-1-acetic acid (Formula V) to form (αS)-α[[(1,1-dimethylethoxy)carbonyl]amino]-3-hydroxytricyclo[3.3.1.1^{3,7}]decane-1-acetic acid (Formula VI)

A preferred method of preparing the free acid (Formula VI) is described in Example 3 of Provisional Application No. 60/561,986 filed Apr. 14, 2004, which is incorporated herein by reference. Alternatively, the following method can be used to make the free acid:

(αS)-(α-Amino-3-hydroxytricyclo[3.3.1.1 ^{3,7}]decane-1-acetic acid (Formula V) (469 g, 2.08 moles) was dissolved in ice cold 1 N NaOH (5 L, 5 moles, 2.4 equiv) in a phase splitter equipped with a temperature probe and a pH probe. THF (2.5 L) was added to the solution. Solid Boc₂O was then added and the reaction mixture was stirred at ambient temperature for approximately 1 hour. EtOAc (4 L) was then added with stirring and the resulting organic and aqueous layers were separated. The pH of the aqueous layer was adjusted to 7 with concentrated HCl. EtOAc (4 L) was then added and additional HCl was added to lower the pH to approximately 1. The total volume of concentrated HCl added was 510 mL. The organic and aqueous layers were again separated and the aqueous layer was extracted with EtOAc (3×3 L). The organic layers were then combined and washed with water (3 L) and brine (3 L). The washed organic layer was then dried with Na₂SO₄ and concentrated on a rotovap at room temperature until dryness. The yield was 542 g of (αS)-α[[(1,1-dimethylethoxy)carbonyl]amino]-3-hydroxytricyclo[3.3.1.1^{3,7}]decane-1-acetic acid (Formula VI).

### D. Coupling Reaction to produce 3-cyano-(αS)-α-(3-hydroxytricyclo[3.3.1.1^{3,7}]dec-1-yl)-β-oxo-(1S,3S,5S)-2-azabicyclo[3.1.0]hexane-2-ethanecarbamic acid, 1,1-dimethylethyl ester (Formula K)

A 2-L three-necked flask equipped with a thermometer, a mechanical stirrer and a gas inlet was charged with (αS)-α[[(1,1-dimethylethoxy)carbonyl]amino]-3-hydroxytricyclo[3.3.1.1^{3,7}]decane-1-acetic acid (Formula VI) (50 g, 153.8 mmol). THF (200 mL) was added and the mixture stirred to produce a clear solution. The solution was cooled to −6° C. in an acetone-dry ice-water bath. Methanesulfonyl chloride (MsCl) (13.1 mL, 169 mmol, 1.1 equiv) was then added in a single portion followed by diisopropylethylamine (94 mL, 539 mmol, 1.1 equiv). The diisopropylethylamine was added slowly over a period of about 4 min to keep the internal temperature below 8° C. The reaction mixture was stirred at 0° C. until all acid was converted to mixed anhydride. (1S,3S,5S)-2-Azabicyclo[3.1.0]hexane-3-carboxamide hydrochloride salt (32.5 g, 200 mmol, 1.1 equiv) and hydroxybenzotriazole (HOBT) (1.04 g, 7.6 mmol, 0.05 equiv) were then added in a single portion and the flask was removed from the cooling bath. The reaction mixture containing compound of Formula K was stirred at room temperature for 2 h and then left overnight at room temperature.

### E. Dehydration and Hydrolysis to Produce 3-cyano-(αS)-α-(3-hydroxytricyclo[3.3.1.1^{3,7}]dec-1-yl)-β-oxo-(1S,3S,5S)-2-azabicyclo[3.1.0]hexane-2-ethanecarbamic acid, 1,1-dimethylethyl ester (Formula L)

Pyridine (6 equiv, 922 mmol, 74.6 mL) was added to the reaction mixture of Part D and the reaction mixture was cooled in a cooling bath to −8° C. Trifluoroacetic anhydride (TFAA) (4 equiv, 616 mmol, 87 mL) was then added slowly over 6 min while keeping the temperature below 10° C. The reaction was stirred at 24° C. for 0.5 h and checked via HPLC (30 μl, 0.5 mL acetonitrile (ACN), 0.5 mL H₂O) for the disappearance of Part D Compound K. The reaction was then cooled in a cooling bath to approximately −3° C. NaOH (5 N, 6 equiv, 0.925 mol, 185 mL) was added to the reaction over 10 min (aqueous pH=9.9) while maintaining the reaction temperature below 10° C. Aqueous K₂CO₃ (319 g, 15 equiv, dissolved in 510 mL H₂O) was added over 5 min (temperature=8° C., aq. pH 11.1). The reaction was allowed to run for 7 h 40 min. The reaction was complete when all intermediates were hydrolyzed to the compound of Formula L as determined via HPLC (30 μl, 0.5 mL ACN, 0.5 mL H₂O).

EtOAc (500 mL) was then added to the reaction mixture and the resulting aqueous and organic layers were separated. The organic layer was washed with 500 mL of buffer solution (2 M H₃PO₄, 1 M NaH₂PO₄). The temperature rose to 23° C. from 15° C. The organic layer was washed with a second 500 mL of buffer solution. The organic layer was washed with 300 mL of brine, 130 mL of sat. NaHCO₃ solution and 300 mL of half sat. brine. Darco (5 g) was added to the organic phase. The mixture was stirred for 5 min and filtered through 50 g of silica gel, which was washed with 4×25 mL EtOAc.

The filtrate was then concentrated to approximately 133 mL. The organic layer was stirred for 1 hour until the solution turned cloudy. Heptane (133 mL) was added over 15 min and the slurry stirred overnight. More heptane (133 mL) was added and mixture was stirred violently for 20 min with mechanical stirring. The solids were filtered off and the cake was washed with 50 mL of 5% EtOAc/heptane. Dry product crystals were heated at 50° C. under vacuum overnight. 467 g Product (Formula L) was obtained in ˜73% yield (46.7 g, 96.6 AP).

### F. Deprotection of L

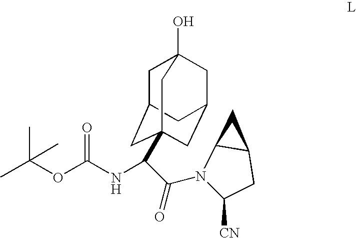 to produce free base M 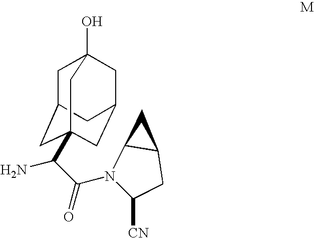
and the corresponding monohydrate M′.

Part E compound (L) (300 g, 0.723 mol, potency of 90.6%), dichloromethane (3 L), methanol (288 mL, 7.23 mol) and concentrated (36%) hydrochloric acid (288 mL, 7.23 mol) were charged to a 3-neck 12-L flask equipped with mechanical stirrer, temperature probe and N₂ gas inlet. Reaction occurred while maintaining reaction temperature within the range from about 20 to about 25 ° C. The reaction mixture was stirred for 18 h, split into 2 phases and the top aqueous layer was collected. The aqueous layer containing the hydrochloric salt 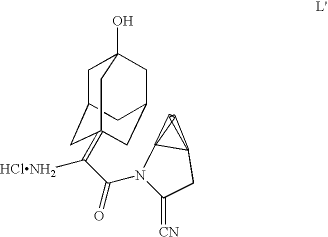
(identified by HPLC) (Formula L′) was treated with dichloromethane (6 L) and water (720 mL), and 5 N sodium hydroxide solution (˜600 mL) was added dropwise while maintaining reaction temperature between 20 and 25° C. to adjust pH between 9 and 10.5. NaCl (120 g) was added and the mixture agitated for 20 min to form a phase split. The organic layer (6.2 L) containing ˜174 g of compound M was collected and the aqueous layer (1.75 L) containing 6.5 g of compound M was discarded.

The organic layer was washed with 1% NH₄Cl brine solution (450 mL; 1 g of NH₄Cl, 25 g of NaCl and 74 g of H₂O per 100 mL). From the resulting phase split, 6.0 L of organic layer containing ˜176 g of compound M was recovered and the aqueous layer (0.45 L) containing 1.4 g of compound M (˜0.4%) was discarded. Ethyl acetate (˜4 L) was added to the organic layer while dichloromethane was distilled off at 25° C./50 mm Hg. Distillation was discontinued when a final volume of 2.5 L was reached. The organic layer was polish filtered to remove solid NaCl and was concentrated to ˜1 Kg (˜170 g of compound M in 1 L ethyl acetate containing <0.1% CH₂Cl₂ by GC analysis). Water (17 mL) was added dropwise and after 10 min crystallization began. Water (17 mL) was added and the resulting slurry was agitated for 30 min, and then filtered. The cake was washed with ethyl acetate and dried at room temperature under vacuum to give 186 g of monohydrate M′, yield 81%.

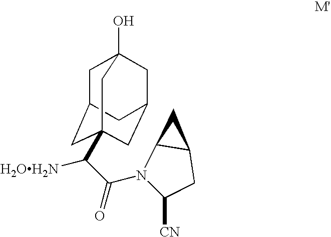

What is claimed is:

1. A process for preparing an intermediate compound of the Formula A 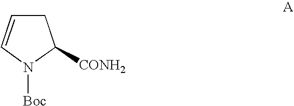
which comprises
a) providing an ester of the Formula B 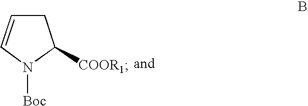
where R₁ is ethyl or methyl
b) reacting ester B with an ammonia source and a base to form the intermediate A.

2. The process as defined in claim 1 wherein the reaction to form the Formula A compound is carried out in a one-pot procedure.

3. The process as defined in claim 1 wherein the reaction is carried out in the presence of a solvent or without solvent.

4. The process as defined in claim 1 wherein the ammonia source is formamide, ammonia gas, ammonium carbamate, ammonium formate, ammonium phosphate, ammonium acetate, ammonium fluoride, ammonium bromide, ammonium chloride, ammonium iodide, ammonium iodate, ammonium carbonate, ammonium citrate, ammonium chromate, ammonium dichromate, ammonium hydroxide, ammonium lactate, ammonium molybdate, ammonium nitrate, ammonium oxalate, ammonium sulfate, ammonium sulfide, ammonium tartrate, ammonium triflate, ammonium thiocyanate, ammonium dihydrogen phosphate, urea, methyl carbamate, ethyl carbamate, propyl carbamate or t-butyl carbamate.

5. The process as defined in claim 1 wherein the base is an alkali metal alkoxide which is a sodium alkoxide, potassium alkoxide, magnesium alkoxide or lithium alkoxide, or an alkali metal methoxide, an alkali metal ethoxide, an alkali metal propoxide or an alkali metal butoxide.

6. The process as defined in claim 1 wherein the base is such as sodium methoxide, potassium methoxide, lithium methoxide, magnesium methoxide, magnesium ethoxide, sodium ethoxide, potassium ethoxide, lithium ethoxide, sodium propoxide, potassium propoxide, lithium propoxide, sodium t-butoxide, potassium t-butoxide, lithium t-butoxide, sodium hydride, potassium hydride, pyridine, triethylamine, diethylamine, diisopropylamine, diisopropylethylamine (Hunig's base), 1,8-diazabicyclo[5.4.0]undec-7-ene (DBU), 1,5-diazabicyclo[4.3.0]non-5-ene (DBN), 1,4-diazabicyclo[2.2.2]octane (DABCO), KHCO₃, NaHCO₃, Na₂CO₃, K₂CO₃, Li₂CO₃, BaCO₃, CaCO₃, CS₂CO₃, MgCO₃, KOH, NaOH, or LiOH, either alone or with catalytic amounts of NiCl₂, CeCl₃, MgBr₂, Sc(III)(OTf)₃, Fe(OAc)₂, Cu(I)SCN, basic alumina, AgOAc, MnCl₂, Cu(OAc)₂, Co(OAc)₂, Zn(OAc)₂, Pd(OAc)₂, FeCl₃, Ti(OPr)₄, tetrabutylammonium chloride, tetrabutylammonium bromide, dodecanethiol or 2-hydroxypyridine.

7. The process as defined in claim 3 wherein the solvent is formamide, dichloromethane, toluene, chloroform, THF, acetonitrile, methyl acetate, ethyl acetate, isopropyl acetate, propyl acetate, butyl acetate, acetone, methyl isobutyl ketone, methyl ethyl ketone, 1,2-dimethoxyethane, 2-methyltetrahydrofuran, 1,4-dioxane, methyl t-butyl ether (MTBE), chlorobenzene, xylenes, heptane, hexanes, cyclohexane, cyclohexanone, DMF, dimethyl sulfoxide, N-methylpyrrolidinone, MTBE, methanol, ethanol, isopropanol, n-propanol, n-butanol, t-butanol or ethylene glycol.

8. The process as defined in claim 1 wherein the ammonia source is employed in a molar ratio to the base within the range from about 1:1 to about 200:1.

9. The process as defined in claim 1 wherein the ammonia source is formamide, the base is sodium methoxide and the solvent is methanol.

10. The process as defined in claim 9 wherein the formamide is employed in a molar ratio to the sodium methoxide within the range from about 1:1 to about 200:1.

11. The process as defined in claim 9 including the steps of
a) dissolving ester B in methanol; and
b) adding the solution of formamide and sodium methoxide in methanol to the solution of ester B in methanol.

12. The process as defined in claim 1 wherein the reaction in step b) is carried out at a temperature within the range from about −100 to about 200° C.

13. The process as defined in claim 1 wherein said compound of Formula A is employed in the preparation of the hydrochloride or mesylate salt of (1S,3S,5S)-2-azabicyclo[3.1.0]-hexane-3-carboxamide (Formula J).

14. The process as defined in claim 1 wherein said compound of Formula A is employed in the preparation of a dipeptidyl peptidase IV inhibitor.

15. A method for preparing a compound of the formula 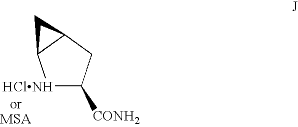
which comprises
a) preparing a compound of Formula A employing the process as defined in claim 1 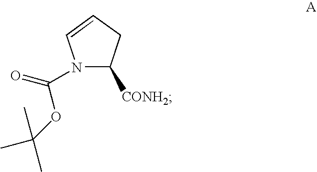
b) subjecting the compound of Formula A to cyclopropanation via a Simmons-Smith reaction to produce a compound of Formula H 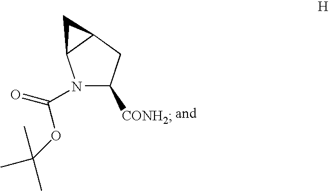
deprotecting the compound of Formula H to form the compound of Formula J.

16. A method for preparing a compound of the Formula M 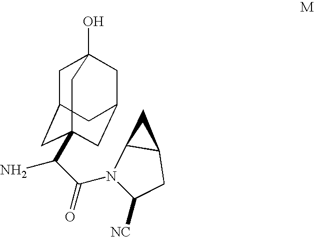
or its monohydrate M′which comprises
a) providing a compound of the Formula J prepared by the method as defined in claim 15 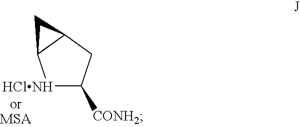
b) coupling the compound of Formula J with compound of the formula VI 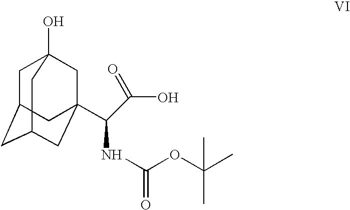
in the presence of mesyl chloride, Hunig's base and 1-hydroxybenzotriazole (HOBT) to form a compound of Formula K 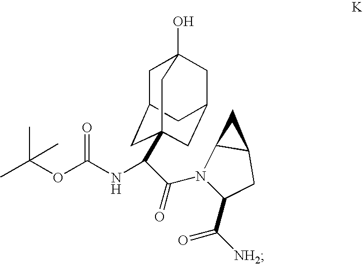
c) dehydrating the compound of Formula K in the presence of pyridine and trifluoroacetic anhydride, and then hydrolyzing the reaction product in the presence of strong base to produce a compound of Formula L 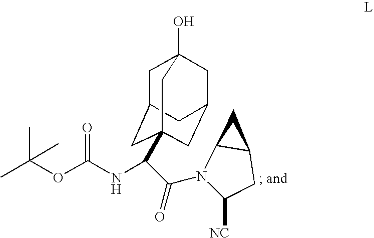
d) deprotecting the compound of Formula L to produce the compound of Formula M.

17. The process as defined in claim 16 wherein deprotecting step d) is carried out by treating compound of formula L with hydrochloric acid to form the corresponding hydrochloric acid salt L′, 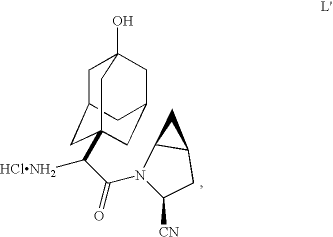
treating compound L′ with sodium hydroxide to form the free base compound M, and treating the free base M with water to form the corresponding monohydrate M′.
